(19) United States
(12) Patent Application Publication
(10) Pub. No.: US 2024/0350121 A1
(43) Pub. Date: Oct. 24, 2024

(54) SYSTEMS AND METHODS FOR THREE-DIMENSIONAL IMAGING

(71) Applicant: INTUITIVE SURGICAL OPERATIONS, INC., Sunnyvale, CA (US)

(72) Inventors: Randall L. Schlesinger, San Mateo, CA (US); Serena H. Wong, Los Altos, CA (US)

(21) Appl. No.: 18/641,144

(22) Filed: Apr. 19, 2024

Related U.S. Application Data
(60) Provisional application No. 63/497,644, filed on Apr. 21, 2023.

(51) Int. Cl. A61B 8/08; A61B 8/00; A61B 8/12; A61B 34/20

(52) U.S. Cl. CPC A61B 8/483; A61B 8/12; A61B 8/4254; A61B 8/4416; A61B 8/4488; A61B 8/4494; A61B 8/54; A61B 34/20

(57) ABSTRACT

A system may comprise an elongate flexible instrument including an imaging device and a localization sensor. The system may capture a first 2D image with the imaging device in a first imaging configuration and receive first localization data for the distal portion of the elongate flexible instrument from the localization sensor while the imaging device is in the first imaging configuration. The system may also create a first image data set including the first localization data and the first 2D image, capture a second 2D image with the imaging device in a second imaging configuration, and receive second localization data for the distal portion while the imaging device is in the second imaging configuration. The system may also create a second image data set including the second localization data and the second 2D image and generate a 3D image based on a plurality of image data sets.

### CROSS-REFERENCED APPLICATIONS

This application claims priority to and benefit of U.S. Provisional Applications No. 63/497,644 filed Apr. 21, 2023 and entitled “Systems and Methods for Three-Dimensional Imaging,” which is incorporated by reference herein in its entirety.

### FIELD

The present disclosure is directed to systems and methods for generating three-dimensional images from two-dimensional images and associated image system localization information.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation and interventional procedures at the region of interest may be assisted using intra-operative images of the anatomic passageways and surrounding anatomy. Improved systems and methods are needed to generate intra-operative images to visualize target anatomic structures during interventional procedures.

### SUMMARY

The following presents a simplified summary of various examples described herein and is not intended to identify key or critical elements or to delineate the scope of the claims.

Consistent with some examples, a system may comprise an elongate flexible instrument including an imaging device disposed at a distal portion of the elongate flexible instrument and a localization sensor within the elongate flexible instrument. The system may also comprise a controller comprising one or more processors configured to capture a first two-dimensional image with the imaging device in a first imaging configuration and receive first localization data for the distal portion of the elongate flexible instrument from the localization sensor while the imaging device is in the first imaging configuration. The one or more processors may also be configured to create a first image data set including the first localization data and the first two-dimensional image, capture a second two-dimensional image with the imaging device in a second imaging configuration, and receive second localization data for the distal portion of the elongate flexible instrument from the localization sensor while the imaging device is in the second imaging configuration. The one or more processors may also be configured to create a second image data set including the second localization data and the second two-dimensional image and generate a three-dimensional image based on a plurality of image data sets, including the first and second image data sets.

Consistent with some examples, a system may comprise an elongate flexible instrument including an imaging device disposed at a distal portion of the elongate flexible instrument and a localization sensor extending within the elongate flexible instrument to the distal portion. The system may also comprise a controller comprising one or more processors configured to capture a first two-dimensional image with the imaging device in a first configuration, receive first localization data for the distal portion of the elongate flexible instrument from the localization sensor while the imaging device is in the first configuration, and create a first image data set including the first two-dimensional image and the first localization data. The one or more processor may also be configured to generate a plurality of image data sets with the imaging device arranged in a plurality of different configurations, the plurality of image data sets including the first image data set and generate a three-dimensional image based on the plurality of image data sets.

Consistent with some examples, a method may comprise capturing a first two-dimensional image with an imaging device in a first configuration, the imaging device disposed at a distal portion of an elongate flexible instrument, receiving first localization data for the distal portion of the elongate flexible instrument from a localization sensor extending within the elongate flexible instrument to the distal portion while the imaging device is in the first configuration and creating a first image data set including the first localization data and the first two-dimensional image. The method may also comprise capturing a second two-dimensional image with the imaging device in a second configuration, receiving second position data for the distal portion of the elongate flexible instrument from the 1 sensor while the imaging device is in the second configuration, creating a second image data set including the second position data and the second two-dimensional image and generating a three-dimensional image based on the first and second image data sets.

Other examples include corresponding computer systems, apparatus, and computer programs recorded on one or more computer storage devices, each configured to perform the actions of the methods.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an example of an elongate instrument in a patient anatomy near a target, according to some examples.

FIG. 2 illustrates an elongated, flexible medical instrument system including a sensing instrument, according to some examples.

FIG. 3 is a flow chart illustrating a method for generating a three-dimensional image based on image data sets, according to some examples.

FIGS. 4A and 4B are image data sets, according to some examples.

FIG. 4C is a three-dimensional image generated from the image data sets of FIGS. 4A and 4B, according to some examples.

FIG. 5A illustrates an elongated, flexible sensing instrument including a side-facing imaging device, according to some examples.

FIG. 5B illustrates a top view of a sensing instrument with a side-facing imaging device, according to some examples.

FIG. 5C illustrates a side view of the sensing instrument of FIG. 5B.

FIG. 6A-6C illustrate image scanning motion for a side-facing imaging device, according to some examples.

FIG. 7 illustrates an elongated, flexible sensing instrument including a forward-facing imaging device, according to some examples.

FIG. 8A and 8B illustrate distal end surface views of sensing instruments with forward-facing imaging devices, according to some examples.

FIG. 9A-9C illustrate an image scanning motion for a forward-facing imaging device, according to some examples.

FIG. 10A-10B illustrate distal end surface views of sensing instruments with forward-facing imaging devices, according to some examples.

FIG. 11A-11B illustrate an image scanning motion for a forward-facing imaging device, according to some examples.

FIG. 12 illustrates an elongated, flexible sensing instrument including a radial imaging device, according to some examples.

FIG. 13A-13C an image scanning motion for a radial imaging device, according to some examples.

FIG. 14 illustrates a graphical user interface that may be displayed during the planning of, navigation to, or conducting of an interventional medical procedure, according to some examples.

FIG. 15 illustrates a graphical user interface that may be displayed during the planning of, navigation to, or conducting of an interventional medical procedure, according to some examples.

FIG. 16 illustrates a simplified diagram of a robot-assisted medical system according to some examples.

FIG. 17A is a simplified diagram of a medical instrument system according to some examples.

FIG. 17B is a simplified diagram of a medical instrument with an extended medical tool according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The techniques disclosed in this document may be used to enhance intra-operative sensing instruments, including intra-operative imaging instruments, and their use in minimally invasive procedures. In some examples, intra-operative sensing data, including imaging data and localization data, may be utilized to generate three-dimensional intraoperative images of target tissue. The sensing instrument may include sensing systems including an imaging system and an imaging localization system. Although some of the imaging systems described herein are ultrasound imaging systems and some of the imaging localization systems described herein are optical fiber shape sensor systems, it is contemplated that the systems and methods described herein may be applied to other imaging and sensing modalities without departing from the scope of the present disclosure.

The systems and techniques described in this document may be used in a variety of medical procedures that may improve accuracy and outcomes through use of intra-operative imaging. For example, intra-operative imaging may be used to biopsy lesions or other tissue to, for example, evaluate the presence or extent of diseases such as cancer or surveil transplanted organs. As another example, intra-operative imaging may be used in cancer staging to determine via biopsy whether the disease has spread to lymph nodes. The medical procedure may be performed using hand-held or otherwise manually controlled imaging probes and tools (e.g., a bronchoscope). In other examples, the described imaging probes and tools may be manipulated with a robot-assisted medical system.

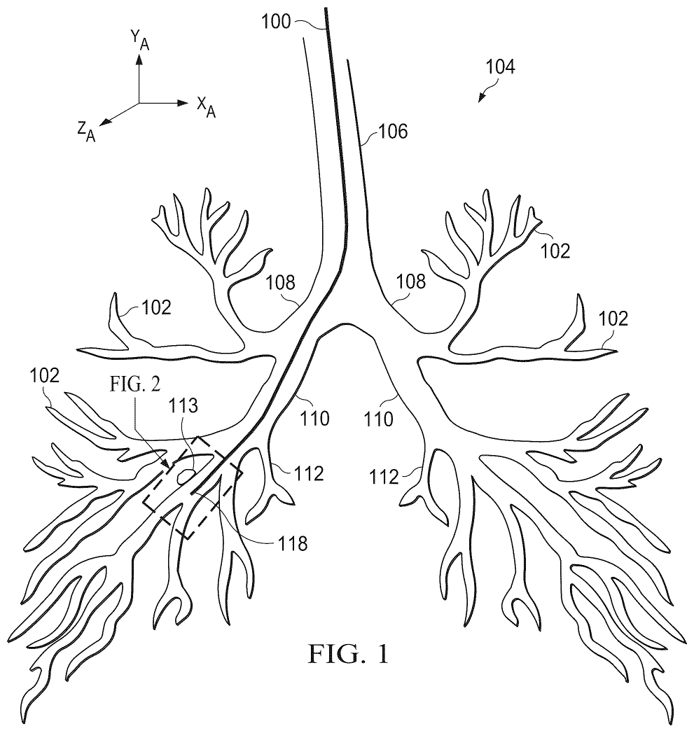

FIG. 1 illustrates an elongated, flexible medical instrument system 100 extending within branched anatomic passageways or airways 102 of an anatomical structure 104. In some examples the anatomic structure 104 may be a lung and the passageways 102 may include the trachea 106, primary bronchi 108, secondary bronchi 110, and tertiary bronchi 112. The anatomic structure 104 has an anatomical frame of reference (X_{A}, Y_{A}, Z_{A}). A distal end portion 118 of the medical instrument system 100 may be advanced into an anatomic opening (e.g., a patient mouth) and through the anatomic passageways 102 to perform a medical procedure, such as a biopsy, at or near a target tissue 113. The target tissue 113 may include a lymph node, a tumor, or other tissue or substance of interest.

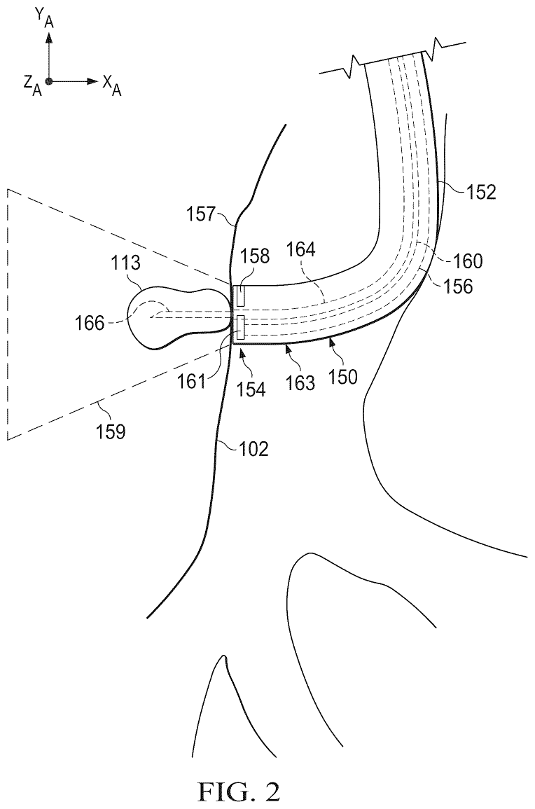

As shown in FIG. 2 an elongated, flexible medical instrument system 150 (e.g., the elongated medical instrument system 100) may include an elongate flexible sensing instrument 152 extendable through the anatomic passageway 102. In some examples, the components of the sensing instrument 152 may be integrated into a manually-controlled bronchoscope or a robot-assisted steerable catheter system such as medical instrument system 800. The sensing instrument 152 may include an imaging system 154 and an imaging localization system 156. In some examples, the imaging system 154 may include an imaging device 161 such as an ultrasound transducer located at a distal end portion 163 of the sensing instrument 152. The imaging system 154 may generate image data for a field of view 159. Ultrasound transducers may include transducer arrays that may be comprised of a plurality of transducers of any size or shape, as described in greater detail below. For ultrasound imaging, contact between the sensing instrument 152 and a wall 157 of the anatomic passageway 102 may be required or may improve the quality of the sensor or imaging data received from the sensing element. For example, if the imaging device 161 is an ultrasound transducer, contact between the sensing instrument 152 and the wall 157 may eliminate air gaps and promote the effective transmission of the ultrasound signal and the generation of a clear image.

In some examples, the imaging localization system 156 may include, for example, a localization sensor such as an optical fiber shape sensor, an electromagnetic (EM) sensor, or plurality of EM sensors positioned at a known location relative to the imaging system 154 to track the position and orientation of imaging system 154. The localization system 156 may be used to track the configuration, including position and orientation, of the distal end portion 163 of the sensing instrument 152, including the imaging device 161, in six degrees of freedom. Thus, the localization data from the localization system 156 may be used to record the configuration of the image data from the imaging device 161 in three-dimensional space. In one example, an optical fiber forms a fiber optic bend sensor for determining the shape of the sensing instrument 152. The optical fiber or a portion of the optical fiber may be fixed at the distal end portion 163 of the sensing instrument 152 or at a known location relative to the imaging device 161 to provide localization data, including position and/or orientation data, for the imaging device 161. With a distal end of the optical fiber shape sensor fixed at a constant offset to the imaging device, the position and orientation of the imaging device may be determined by the measured shape of the sensor. A proximal end of the shape sensor may be fixed or known relative to a robot-assisted medical system. In other examples, if the sensing instrument is manually manipulated, the proximal end of the shape sensor may be fixed to the patient body or another fixed or tracked location near the patient.

Optical fibers including Fiber Bragg Gratings (FBGs) may be used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fiber Bend Sensor”), which are all incorporated by reference herein in their entireties. Sensors in some embodiments may employ other suitable strain sensing techniques, such as Rayleigh scattering, Raman scattering, Brillouin scattering, and Fluorescence scattering. In some examples, the shape of the localization of the imaging system 154 may be determined using other techniques. For example, a history of the distal end pose of sensing instrument can be used to reconstruct the shape of sensing instrument over the interval of time. In some embodiments, a shape sensor may comprise a plurality of position sensors (such as electromagnetic position sensors) which collectively provide shape data regarding a shape of at least a portion of the sensing instrument. It should be appreciated that “shape sensor” as used herein may refer to any suitable localization sensor. Generally, a shape sensor as that term is used herein may provide any number of data points in any number of degrees of freedom including three or six degrees of freedom at a series of points monitored by the shape sensor along the length of the elongate instrument.

In some examples, the sensing instrument 152 may include a steering system 160, including control wires, cables, or other control apparatus to bend or steer a distal end portion of the sensing instrument, which may include the imaging system 154. In some examples, the sensing instrument 152 may also include a channel or passage 164 through which an interventional tool 166 may be extended to emerge from a distal or side port of the sensing instrument 152 to engage the target tissue 113. The interventional tool 166 may include, for example, a biopsy or tissue sampling tool, an ablation tool including a heated or cryo-probe, an electroporation tool, a forceps, a medication delivery device, a fiducial delivery device, or another type of diagnostic or therapeutic device. In some examples, the interventional tool may have a flexible shaft. The interventional tool may include control wires or other control apparatus to bend or steer the direction of the interventional tool. Since it may be beneficial to provide real time visualization of the interventional tool positioned within or near a target tissue, the interventional tool may be delivered within the imaging field view of view of an ultrasound imaging instrument (e.g. instrument 152) for direct visualization of the interventional tool into the target tissue 113. The sensing instrument 152 may, optionally, include other sensing systems such as a visible light optical imaging system 158 positioned at a distal end portion of the sensing instrument 152.

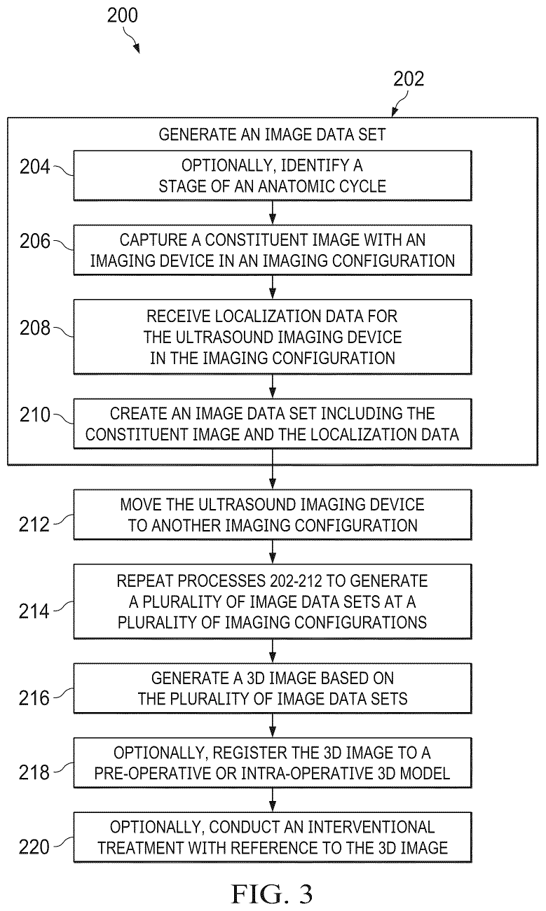

FIG. 3 is a flow chart illustrating a method 200 for generating a three-dimensional image based on image data sets from a sensing instrument. The method 200 is illustrated as a set of operations or processes that may be performed in the same or in a different order than the order shown. One or more of the illustrated processes may be omitted in some examples of the method. Additionally, one or more processes that are not expressly illustrated in FIG. 3 may be included before, after, in between, or as part of the illustrated processes. In some examples, one or more of the processes of method 200 may be implemented, at least in part, by a control system executing code stored on non-transitory, tangible, machine-readable media that when run by one or more processors (e.g., the processors of a control system) may cause the one or more processors to perform one or more of the processes

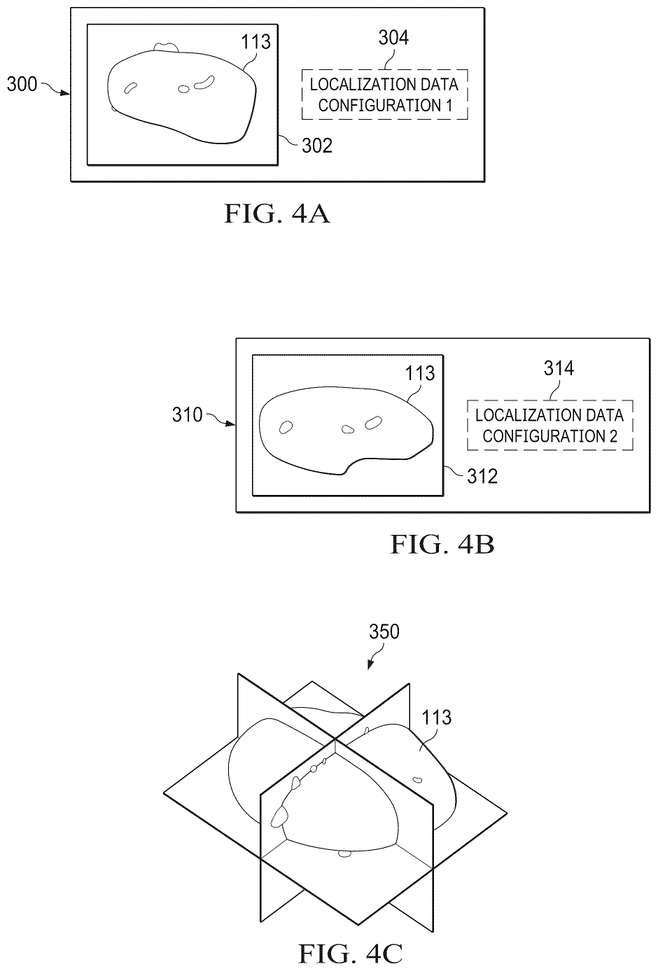

At a process 202, an image data set may be generated. An image data set may include a constituent image of a composite image and a set of localization information associated with the constituent image. In some examples, the image data set may include image-only data. In other examples, the image data set may include localization information per image. In some examples, some image data sets used in the composite image may include localization data and others may include image-only data. As explained in process 214, one or more subprocesses of process 202 may be repeated to generate a plurality of image data sets. FIG. 4A illustrates an example of a first image data set 300 with an imaging device (e.g., transducer 161) at an initial configuration, and FIG. 4B illustrates an example of a second image data set 310 with an imaging device (e.g., transducer 161) at a second configuration.

In some examples, each image data set used in the generation of a composite image may be generated at a periodic stage of an anatomic cycle. Thus, at an optional process 204, the periodic stage of an anatomic cycle may be identified. The capture of each constituent image data set used to form a composite image may be gated or confined to the same stage of the identified anatomic cycle. The anatomic cycle may be, for example, a cardiac or respiratory cycle. For example, each image data set used in the generation of a composite image may be gathered at a gated stage of a respiratory cycle, such as full inhalation or full exhalation. Alternatively, if the patient's breathing is under control of a respirator, the capture of the constituent image data set may be performed under a breath hold controlled by the respirator.

At a process 206, a constituent image may be captured with a sensing instrument while the sensing instrument is arranged in an initial configuration. For example, a constituent image may be a two-dimensional ultrasound image of the target tissue 113 captured by the imaging device 161 (e.g., an ultrasound transducer) of the sensing instrument 152 while the imaging device is located in an initial configuration within the passageway 102. In the example of FIG. 4A, the image data set 300 includes a constituent image 302 which is a two-dimensional ultrasound image of the target tissue 113 taken with the imaging device 161 in an initial configuration (e.g., Configuration 1).

At a process 208, localization data for the sensing instrument may be received, recorded, or otherwise captured while the imaging device is located in the initial configuration. For example, the imaging localization system 156 may capture localization data, including, for example, position and/or orientation data while the imaging device 161 is located in the initial configuration within the passageway 102. In the example of FIG. 4A, the image data set 300 includes a localization data set 304 captured with the imaging device 161 in an initial configuration (e.g., Configuration 1). The localization data set 304 may be optical fiber shape sensor information (including any offset between the shape sensor and the imaging device) that provides the position and/or orientation of the imaging device 161, and thus the constituent image 302, in three-dimensional space.

At a process 210, an image data set may be created including the constituent image and the localization data. For example, an image data set may include the two-dimensional ultrasound image of the target tissue 113 generated by the sensing instrument 152 with the imaging device 161 is at the initial position and orientation and may include the associated localization data for the imaging device 161 at the initial position and orientation. In the example of FIG. 4A, the image data set 300 includes the constituent image data 302 and the localization data set 304 captured with the imaging device 161 in an initial configuration (e.g., Configuration 1).

At a process 212, the sensing instrument may be moved from the initial configuration to a different imaging configuration. For example, the distal end portion 163 of sensing instrument 152 may be moved from the initial configuration to a second configuration to change a position and/or orientation of the imaging device 161. More specifically, the distal end portion 163 of the sensing instrument 152 may be translated (e.g. in an X_{A}, Y_{A}, and/or Z_{A} direction) or rotated (e.g., about any of the axes X_{A}, Y_{A}, Z_{A}) in any of six degrees of freedom to change the configuration and field of view of the imaging device 161.

At a process 214, with the sensing instrument moved to another configuration, the processes 202-212 may be repeated one or more times to generate a plurality of image data sets, each at a different imaging configuration. For example, the distal end portion 163 of the sensing instrument 152 may be moved through a series of configurations, collecting an image data set, including a two-dimensional ultrasound image and a set of associated localization data, at each configuration in the series of configurations. In the example of FIG. 4B, the image data set 310 includes the constituent image data 312 and a localization data set 314 captured with the imaging device 161 in a second configuration (e.g., Configuration 2). A plurality of image data sets may include the image data sets 300, 310 and/or other image data sets gathered with the distal end portion 163 of the sensing instrument 152 and the imaging device 161 in various configurations near the target tissue 113.

At a process 216, a three-dimensional image may be generated from the plurality of image data sets. For example, the two-dimensional constituent images may be stitched together with imaging software to generate a three-dimensional composite image. The image stitching algorithms may utilize the localization data from each of the image data sets with feature detection and matching from the two-dimensional images to register, align, calibrate, and blend the two-dimensional constituent images to form the three-dimensional composite image. FIG. 4C illustrates a three-dimensional image 350 generated from the image data sets 300, 310. The image 350 may be displayed on a display device and may be manipulated in, for example, six degrees of freedom to allow a clinician to fully view the size, shape, and features of the target tissue 113. In some examples, after imaging stitching is used to generate a three-dimensional composite image, segmentation and other calculations or processing may be performed to create a mesh structure that may allow for better visualization and measurements of the target tissue.

At an optional process 218, the three-dimensional composite image may be registered to a three-dimensional, pre-operative or intra-operative model. Pre-operative and/or intra-operative image data may be captured and used to generate a three-dimensional model using imaging technology such as computerized tomography (CT), cone-beam CT, magnetic resonance imaging (MRI), fluoroscopy, tomosynthesis, thermography, optical coherence tomography (OCT), thermal imaging, impedance imaging, laser imaging, nanotube X-ray imaging, and/or the like. For example, a CT scan of the patient's anatomy may be performed pre-operatively or intra-operatively and the resulting image data may be used to construct a segmented 3D model. The 3D CT model may be registered with the three-dimensional composite image so that information such as annotations, navigation routes, identified target structures, identified sensitive tissues, or other structures and features identified, highlighted, or otherwise noted on the 3D CT model may be transferred or associated with the three-dimensional composite image. If the sensing instrument is integrated with a catheter of a robot-assisted medical system that has been registered to the patient anatomy and 3D CT model, the registration of three-dimensional composite image may be registered 3D CT model based on the registration of the catheter. If the sensing instrument is integrated with a manual catheter such as a manual bronchoscope, the proximal end of the shape sensor of the sensing instrument may be fixed to or near the patient or may be trackable (e.g., with encoders, optical fiducials, shape sensors, and/or the like) relative to the patient.

At an optional process 220, an interventional procedure may be conducted with reference to the three-dimensional composite image. In some examples, the three-dimensional composite image may be displayed to a clinician for use in better understanding the configuration of the target tissue, the surrounding structures, possible directions of approach, or other considerations for planning an interventional procedure. In some examples, the three-dimensional image may be displayed with a real-time optical and/or ultrasound image to assist the interventional procedure. In some examples, the three-dimensional image may be displayed with (including adjacent to, overlayed with, or merged with) the registered pre-operative or intra-operative image (e.g., CT image or model). In some examples, markers may be overlayed and stored with the three-dimensional image to track the locations where an interventional procedure (e.g., a biopsy or ablation) occurred. In some examples, the surrounding structures may include blood vessels or other vasculature structures which may be imaged using ultrasound or be part of the three-dimensional ultrasound composite image. The images of the vasculature together with the target tissue may help the clinician avoid damaging the vasculature while performing a procedure.

An interventional procedure may be performed, for example, using the interventional tool 166 which may be, for example, a biopsy or tissue sampling tool, an ablation tool including a heated or cryo-probe, an electroporation tool, a forceps, a medication delivery device, a fiducial delivery device, or another type of diagnostic or therapeutic device. In some examples, the three-dimensional composite image may be used to evaluate the size and shape of the target tissue 113 and to plan multiple locations for performing biopsies to gather sample tissue for evaluation. In some examples, a user-selected virtual interventional path for the interventional instrument may be displayed with the three-dimensional composite image. The interventional instrument may be aligned with the virtual interventional path. A real-time two-dimensional ultrasound image may be displayed with or overlayed on the three-dimensional composite image during the interventional procedure to help guide the intervention, such as a biopsy. Virtual markers may be placed on the three-dimensional image to mark the location of interventional areas, such as biopsy locations.

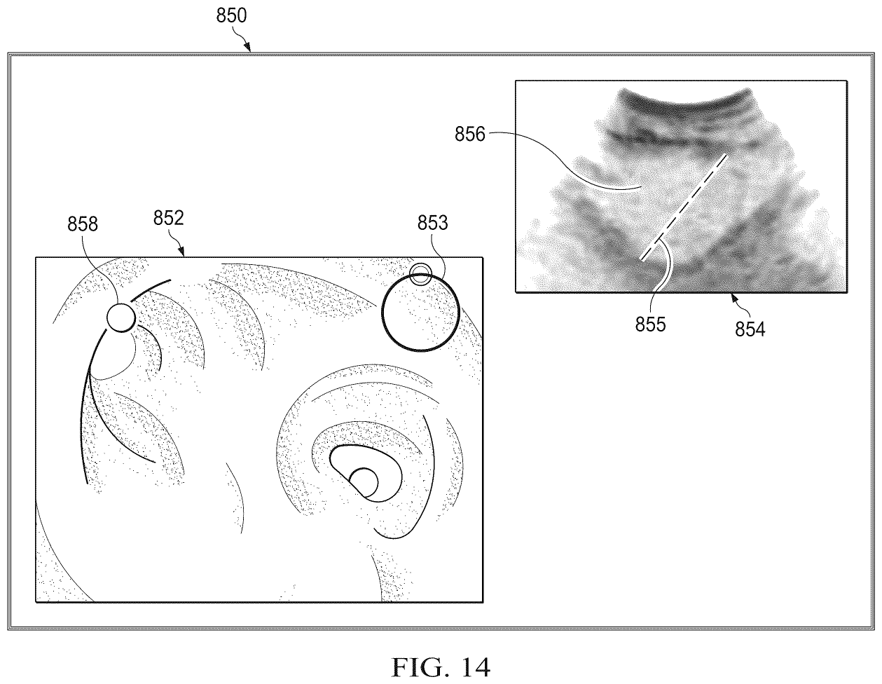

In various examples, supplemental images may be provided to a clinician to guide or assist with positioning an interventional tool and/or previewing an activity. The supplemental images may be two-dimensional guidance images used, for example, in planning, navigation, or conducting an interventional procedure. Further, the supplemental images may be used to supplement a registered three-dimensional pre-operative model (e.g., a pre-operative CT model), a three-dimensional composite image, or other models or images that relate to the procedure. FIG. 14 illustrates a graphical user interface 850 that includes, for example, a two-dimensional guidance image that may be displayed (e.g. on a display system 710) during the planning of, navigation to, or conducting of an interventional medical procedure. The graphical user interface 850 may include an endoscopic or synthetic image 852 of a portion of the patient anatomy and/or a representation image 854 that provides an example or ideal intra-operative image (e.g., a two-dimensional ultrasound image) of target tissue (e.g. target tissue 113). The image 852 may include an indicator 853 of imager (e.g., ultrasound imager) position and/or orientation. The position and/or orientation may be identified using, for example, an optical fiber shape sensor, an electro-magnetic localization sensor, encoders, or other sensors on a rotation mechanism at a proximal end of the interventional tool. Examples of determining the imager orientation and/or position were previously described, for example, with reference to operation 208 and will be described in further detail below. In some examples, the representation image 854 may be reconstructed from the three-dimensional model derived, for example, from CT images in which blood vessels, anatomical structures, and lymph nodes (if visible) may be graphically segmented. In the example of FIG. 14, an interventional procedure may be conducted at a thoracic lymph node station. The synthetic image 852 may be a synthetic image of anatomic passageways near the lymph node station with the location of a node 856 indicated by an indicator 858. The synthetic image 852 may a be a cross-section generated from a portion of a three-dimensional pre-operative model or a three-dimensional composite image. The representation image 854 may be an example or idealized ultrasound image or cut-plane (e.g., a cross-section of the three-dimensional model or image) that provides a clinician with an expected view of the node 856. The representation image 854 may guide the clinician as the clinician performs an intra-operative ultrasound procedure by moving the ultrasound tool until an image similar to the representation image 854 is generated in a real-time image. The graphical user interface 850 may provide guidance for the positioning or trajectory of an interventional tool (e.g. tool 166) by displaying a line 855 or other marker in real-time or virtual two-dimensional views. In some examples, if a biopsy is performed at the node station, biopsy markers may be deposited at the location of each biopsy pass and stored with the three-dimensional model or composite image for future reference.

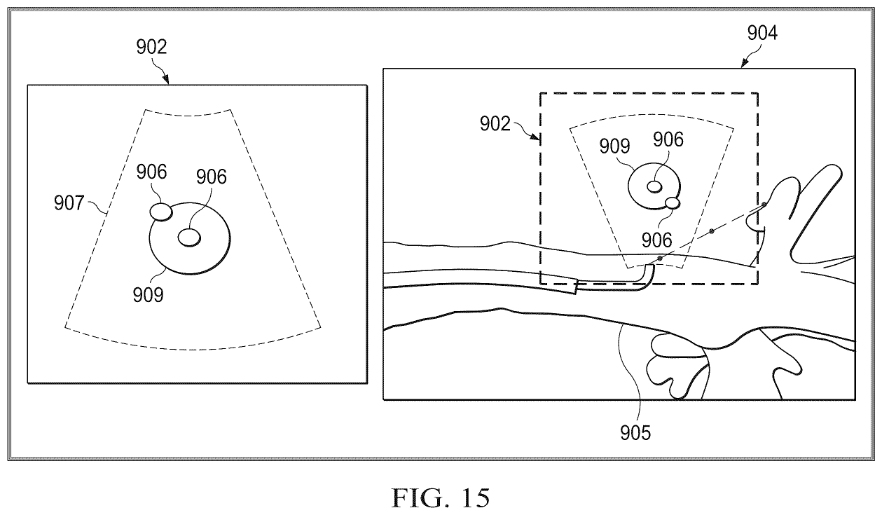

FIG. 15 illustrates a graphical user interface 900 that may be displayed (e.g. on a display system 710) during the planning, navigation, or conducting interventional medical procedure to provide guidance in positioning an interventional tool (e.g. tool 166). The graphical user interface 900 may include an intra-operative image 902 (e.g. an intra-operative ultrasound image) and a composite image 904 that includes all or a portion of the image 902 projected to a three-dimensional image 905 generated from a three-dimensional pre-operative model (e.g., a pre-operative CT model), a three-dimensional composite image, or other models or images that relate to the procedure. For example, the intra-operative image 902 may be a two-dimensional, intra-operative ultrasound image depicting a real-time ultrasound instrument field of view 907 including a lesion 909. The location and orientation of the two-dimensional image may be known based on data recorded by an imaging localization system (e.g., imaging localization system 156) and may be known relative to the registered three-dimensional image space. A clinician may label or mark specific anatomical aspects in the image 902 with reference markers 906 or other annotations via interaction with a user interface (e.g., master assembly 706 and/or display system 710). The three-dimensional image 905 may be from a portion of a pre-operative three-dimensional model of the patient anatomy. The composite image 904 of the images 902 and 905 may be generated by utilizing the tracked position and/or orientation of the tool (e.g., using EM or shape sensing) in space to identify where the tool is in three-dimensional space in relation to the model. The markers 906 may be displayed with the two-dimensional image and also projected to three-dimensional image space by knowing the location of the marker with reference to the two-dimensional image and applying the transformation of the imaging transducer on the interventional tool to obtain global three-dimensional coordinates.

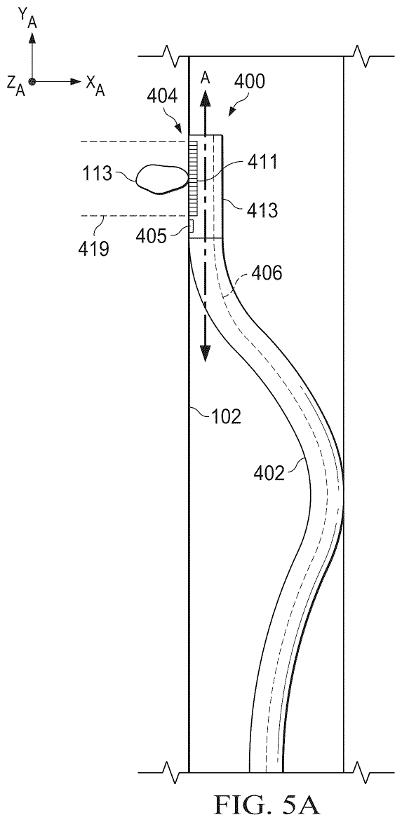

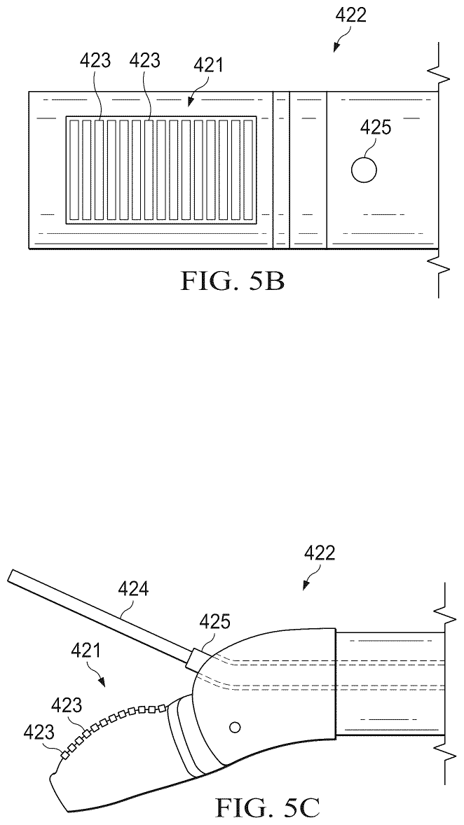

As shown in FIGS. 5A-13C, imaging devices may be provided in a variety of architectures, and various scanning or movement methods may be utilized to capture the constituent images and build the composite three-dimensional image.

FIG. 5A illustrates an elongated, flexible medical instrument system 400 (e.g., the elongated medical instrument system 100, 150) including a sensing instrument 402 extended within an anatomic passageway 102. The sensing instrument 402 may be similar to the sensing instrument 152 with the differences as described. In this example, the sensing instrument 402 includes an imaging system 404 and an imaging localization system 406. The imaging system 404 includes a side-facing imaging device 411 which, in this example is a side-facing ultrasound transducer located at a distal end portion 413 of the sensing instrument 402. A port 405 may allow an interventional tool to be extended from the sensing instrument 402. The imaging system 404 may generate image data for a field of view 419. The imaging device 411 may include a linear array of ultrasound transducer elements extended along a side wall or face of the sensing instrument 152. In some examples, the linear array may be a phased linear array of ultrasound transducer elements. In some examples the imaging localization system 406 may include an optical fiber shape sensor that provides position and orientation information for the distal end portion 413. In other examples, the imaging localization system 406 may include one or more EM sensors.

In some alternative examples, a side-facing imaging device may include a side-facing curved phased array of ultrasound transducer elements. FIG. 5B illustrates a top view and FIG. 5C illustrates a side view of an imaging device 421 of sensing instrument 422 with a curved array of ultrasound transducer elements 423. In this example an interventional tool 424 may extend from a port 425 proximal of the imaging device 421. In some examples, a side-facing imaging device may include a single crystal radial ultrasound transducer that may be spun about the axis A. In some examples, the side-facing imaging device may be exposed through an aperture in a housing surrounding the imaging device.

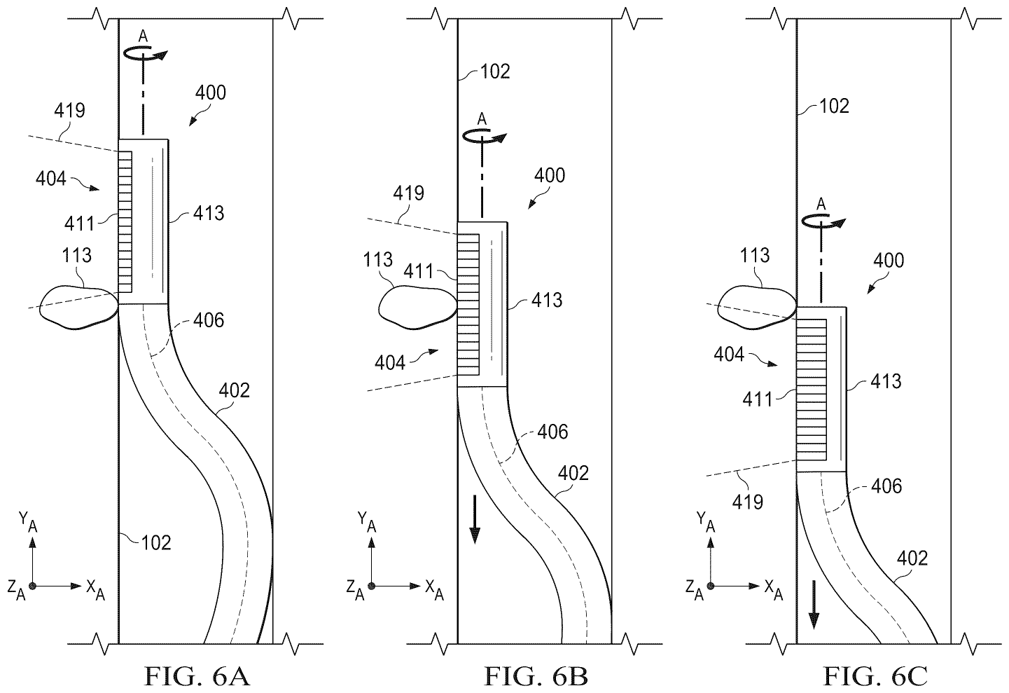

As shown in FIGS. 6A-6C, constituent two-dimensional images used to create a composite three-dimensional image may be generated from the sensing instrument 402 in a series of image capture configurations. As shown in FIG. 6A, the distal end portion 413 and the imaging device 411 of the sensing instrument 402 may be located at a first longitudinal position within the passageway 102 and at a first rotational configuration along a longitudinal axis A (e.g. in a Y_{A} direction). While at the first longitudinal position, a plurality of image data sets may be obtained by capturing image data from the imaging device 411 and localization data from the shape sensor of the imaging localization system 406, while the distal end portion 413, including the imaging device 411, is rotated through a series of rotational positions about the longitudinal axis A. In some alternatives, the imaging device may be rotated relative to stationary portions of the distal end portions. As shown in FIG. 6B, the distal end portion 413 of the sensing instrument 402 may be translated in a generally −Y_{A} direction to a second longitudinal position within the passageway 102. While at the second longitudinal position, a plurality of image data sets may be obtained by capturing image data and localization data while the distal end portion 413, including the imaging device 411, is rotated through a series of rotational positions about the longitudinal axis A. As shown in FIG. 6C, the distal end portion 413 of the sensing instrument 402 may be translated further in a generally −Y_{A} direction to a third longitudinal position within the passageway 102. While at the third longitudinal position, a plurality of image data sets may be obtained by capturing image data and localization data while the distal end portion 413, including the imaging device 411, is rotated through a series of rotational positions about the longitudinal axis A. The translation of the distal end portion 413 and the imaging device 411 may continue until the full target tissue site and any margin areas are imaged. The image data sets obtained at the various longitudinal positions and rotational orientations may be used to generate a three-dimensional image of the target tissue 113 and surrounding anatomic area. More specifically, the two-dimensional images obtained at the various longitudinal positions and rotational orientations may be stitched together based at least on the localization data captured for each of the configurations to generate a three-dimensional image of the target tissue 113 and the surrounding anatomic area.

In other examples, the movement of the distal end portion 413 may be in another translational direction along the wall of the passageway 102, for example the +Y direction or a Z direction. In some examples, the motion of the distal end portion 413, including the distal end portion 413 may be achieved by movement of a control cable (e.g., a control cable of the steering system 160). In some examples a rotational degree of freedom of motion may be provided by manual or robot-assisted control. For example, the motion of the distal end portion may be a 180 degree reciprocating rotational motion. In some examples, translation and rotation may be combined or performed simultaneously. If the target tissue 113 can be imaged from other nearby passageways (e.g., the target tissue is located near a passageway bifurcation), the sensing instrument 402 may be moved to the nearby airway to obtain additional image constituent image data sets that may be used to form a composite three-dimensional image.

In various alternative examples, the sensing instruments described herein, such as the sensing instrument 402, may be slidable through a working channel of a delivery catheter and extendable from a distal opening of the delivery catheter (e.g., the elongate device 802). The sensing instrument may also be withdrawn proximally and removed from the delivery catheter. In examples in which the sensing instrument is separable from a delivery catheter, each of the sensing instrument and delivery catheter may include a shape sensor (e.g., an optical fiber shape sensor). The two shape sensors may be commonly referenced, allowing images generated with data from the sensing instrument to be located relative to the delivery catheter. As the sensing instrument is extended from a delivery catheter, the translational motion and rotational motion of the sensing instrument may be relative to the delivery catheter. For example, movement of the sensing instrument in a −Y direction may cause the sensing instrument to be withdrawn into the delivery catheter, and movement of the sensing instrument in a +Y direction may cause the sensing instrument to extend away from a distal end of the delivery catheter. To register the three-dimensional composite image generated by the sensing instrument to a pre-operative 3D CT model, delivery catheter of a robot-assisted medical system may be first registered to the 3D CT model. The sensing instrument, separable from the delivery catheter, may then be registered to the shape sensor of the delivery catheter.

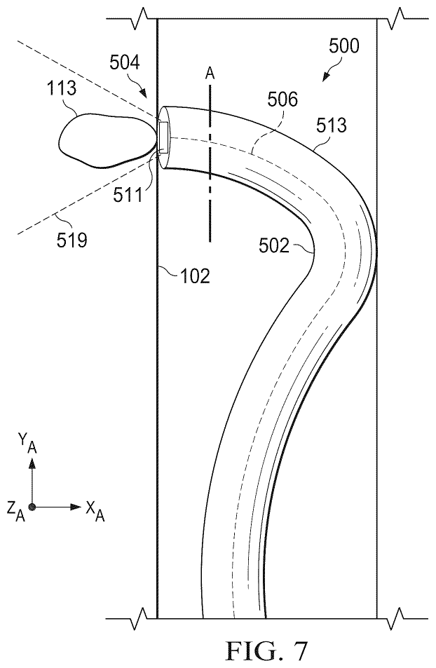

FIG. 7 illustrates an elongated, flexible medical instrument system 500 (e.g., the elongated medical instrument system 100, 150) including a sensing instrument 502 extended within an anatomic passageway 102. The sensing instrument 502 may be similar to the sensing instrument 152 with the differences as described. In this example, the sensing instrument 502 includes an imaging system 504 and an imaging localization system 506. The imaging system 504 includes a forward-facing imaging device 511 which, in this example is a forward-facing ultrasound transducer located on a distal end surface of the distal end portion 513 of the sensing instrument 502. The imaging system 504 may generate image data for a field of view 519. The imaging device 511 may include a linear array of ultrasound transducer elements. In some examples, the linear array may be a phased linear array of ultrasound transducer elements. In this example the imaging localization system 506 may include an optical fiber shape sensor that provides position and orientation information for the distal end portion 513.

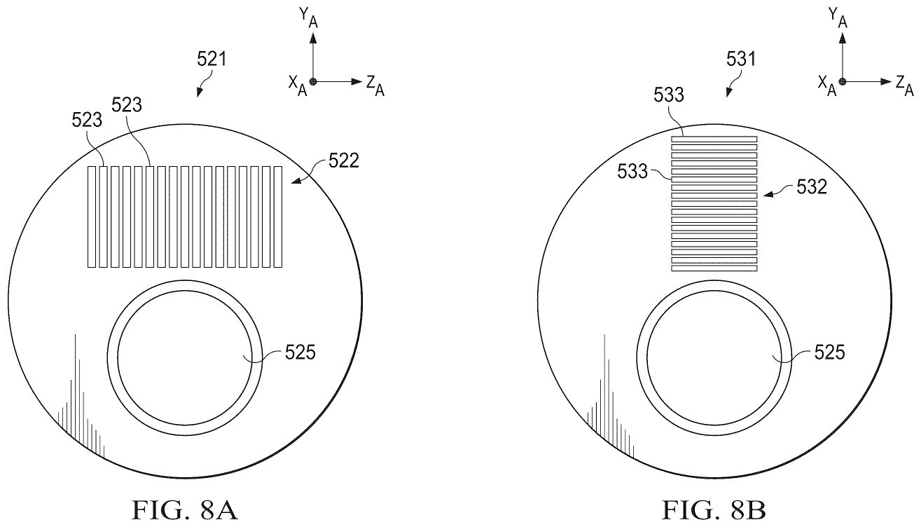

In some examples, a forward-facing imaging device (e.g., imaging device 511) may include a linear array of ultrasound transducer elements. FIG. 8A illustrates a forward-facing imaging device 522 including a linear array of ultrasound transducer elements 523 extending in a generally Z_{A} direction. FIG. 8B illustrates a forward-facing imaging device 532 including a linear array of ultrasound transducer elements 533 extending in a generally Y_{A} direction. In these examples an interventional tool may extend generally in a direction X_{A} from a port 525 near the transducer arrays. In these examples, the transducer arrays 522, 532 may be offset from the port 525. In other examples, transducer arrays may be centered or may surround the port 525. In other examples, multiple forward facing transducer arrays may be arranged around the port 525.

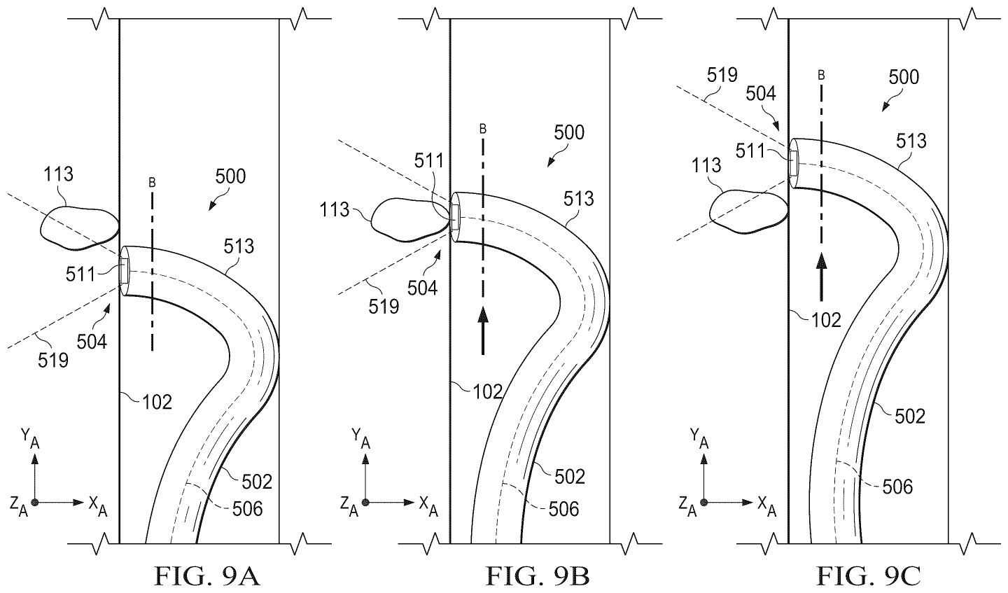

As shown in FIGS. 9A-9C, constituent two-dimensional images used to create a composite three-dimensional image may be generated from the sensing instrument 502 in a series of image capture configurations. In the examples of FIGS. 9A-9C, the imaging device 511 may have a linear array as in FIG. 8A or 8B. As shown in FIG. 9A, the distal end portion 513 and the imaging device 511 of the sensing instrument 502 may be located at a first longitudinal position within the passageway 102 and at a first rotational configuration along a longitudinal axis B (e.g. in a Y_{A} direction). While at the first longitudinal position, a plurality of image data sets may be obtained by capturing image data from the imaging device 511 and localization data from the shape sensor of the imaging localization system 506 at different imaging configurations, while the distal end portion 513, including the imaging device 511, moves through a series of generally Z_{A}-direction translation positions along the curved passageway wall. As shown in FIG. 9B, the distal end portion 513 of the sensing instrument 502 may be translated in a generally +Y_{A} direction to a second longitudinal position within the passageway 102. While at the second longitudinal position, a plurality of image data sets may be obtained by capturing image data and localization data at different imaging configurations while the distal end portion 513, including the imaging device 511, moves through a series of generally Z_{A}-direction translation positions along the curved passageway wall. As shown in FIG. 9C, the distal end portion 513 of the sensing instrument 502 may be translated further in a generally +Y_{A} direction to a third longitudinal position within the passageway 102. While at the third longitudinal position, a plurality of image data sets may be obtained by capturing image data and localization data at different imaging configurations while the distal end portion 513, including the imaging device 511, moves through a series of generally Z_{A}-direction translation positions along the curved passageway wall. The translation of the distal end portion 513 and the imaging device 511 may continue until the full target tissue site and any margin areas are imaged. The image data sets obtained at the various longitudinal positions and rotational and/or translational orientations may be used to generate a three-dimensional image of the target tissue 113 and surrounding anatomic area. More specifically, the two-dimensional images obtained at the various longitudinal positions and rotational orientations may be stitched together based at least on the localization data captured for each of the configurations to generate a three-dimensional image of the target tissue 113 and the surrounding anatomic area.

In other examples, the movement of the distal end portion 513 may be in another translational direction along the wall of the passageway 102, for example the −Y direction or a Z direction. In some examples, the motion of the distal end portion 513, including the distal end portion 513 may be achieved by movement of a control cable (e.g., a control cable of the steering system 160). In some examples a dedicated control cable may actuate the rotational degree of freedom of motion. In some examples, motion of the distal end portion may be a reciprocating rotational motion. In some examples, translation and the rotation may be combined or performed simultaneously. If the target tissue 113 can be imaged from other nearby passageways (e.g., the target tissue is located near a passageway bifurcation), the sensing instrument 502 may be moved to the nearby airway to obtain additional image constituent image data sets that may be used to form a composite three-dimensional image.

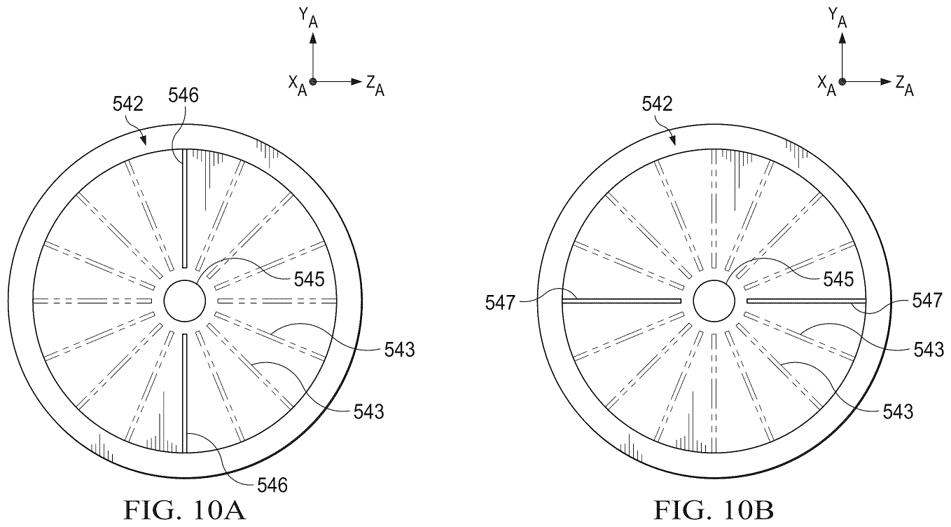

In some examples, a forward-facing imaging device (e.g., imaging device 511) may include an annular array of ultrasound transducer elements. FIGS. 10A and 10B illustrates a forward-facing imaging device 542 including an annular array 543 of ultrasound transducer elements extending radially from around a port 545. An interventional tool may extend generally in a direction X_{A} from the port 545. The ultrasound transducers may generate images in different rotational image planes. For example, transducers 546 may generate a two dimensional image in a first rotational plane, and transducers 547 may generate a two dimensional image in a second rotational plane, different from the first rotational plane.

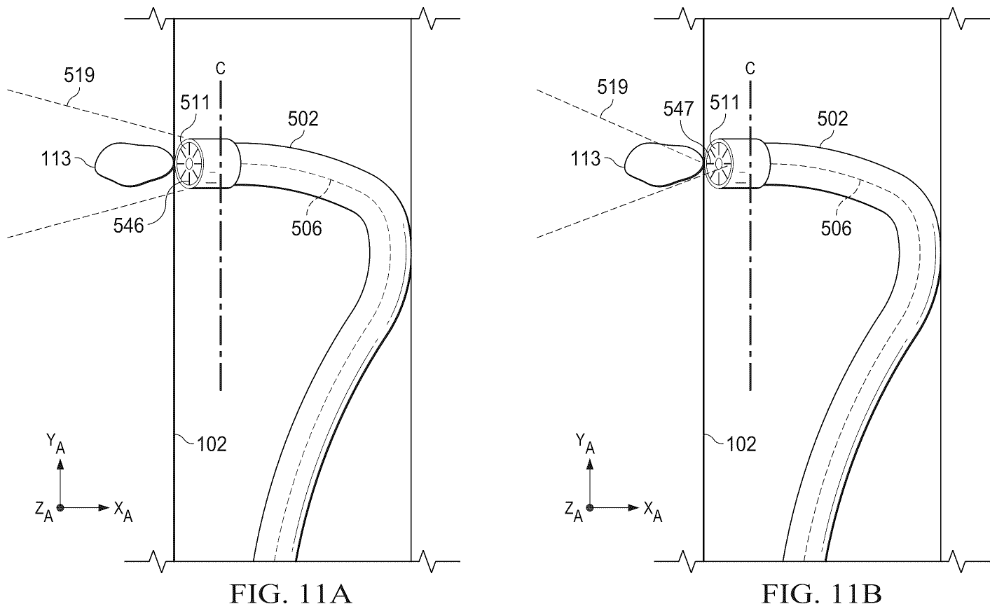

As shown in FIGS. 11A and 11B, constituent two-dimensional images used to create a composite three-dimensional image may be generated from the sensing instrument 502 in a series of image capture configurations. In the examples of FIGS. 11A and 11B, the imaging device 511 may have a radial transducer array as in FIG. 10A and 10B. As shown in FIG. 11A, the distal end portion 513 and the imaging device 511 of the sensing instrument 502 may be located at a first longitudinal position within the passageway 102 and at a first rotational configuration along a longitudinal axis C (e.g. in a Y_{A} direction). While at the first longitudinal position, a plurality of image data sets may be obtained by capturing two-dimensional image data from the imaging device 511 and localization data from the shape sensor of the imaging localization system 506 (e.g. that provides position and orientation data for the distal end portion) and from the imaging device (e.g., that provides image plane rotation information), while the imaging plane of the annular array is electronically rotated through different imaging configurations. For example, as shown in FIG. 11A, a first image data set may be captured at a first image plane by the transducers 546. As shown in FIG. 11B, a second image data set may be captured at a second image plane by the transducers 547. The position and orientation of the distal end portion 513 (measured, for example, by the shape sensor) may remain stationary relative to the passageway 102, but the image plane may be electronically rotated based on the activation of the annular transducers. The captured localization data for each image data set may include information about the orientation of the image plane based on the transducers in the annual array activated to generate a given image. The direction of rotation of the image plane may depend on the order of the fired transducers.

In some examples, if electronically rotating the image plane does not sufficiently image the full area of interest, the distal end portion 513 of the sensing instrument 502 may be translated or pivoted in a generally Y or Z direction (e.g., along or about an axis C) to a second position within the passageway 102. While at the second longitudinal position, a plurality of image data sets may be obtained by capturing image data and localization data as the annular array electronically rotates the image plane at the second position.

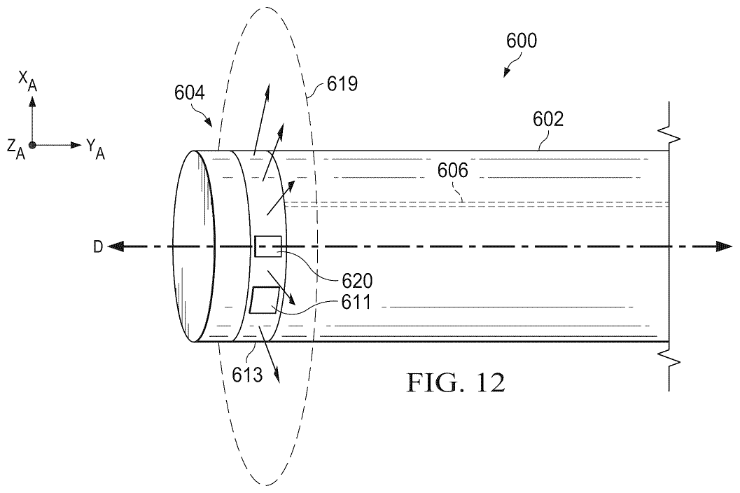

FIG. 12 illustrates an elongated, flexible medical instrument system 600 (e.g., the elongated medical instrument system 100, 150) including a sensing instrument 602. The sensing instrument 602 may be similar to the sensing instrument 152 with the differences as described. In this example, the sensing instrument 602 includes an imaging system 604 and an imaging localization system 606. The imaging system 604 may include a radial imaging device 611 which may be, for example, a radially spinning ultrasound transducer or crystal located at a distal end portion 613 of the sensing instrument 602. In other examples, the radial imaging device 611 may be a solid state radial device with ultrasound elements arranged in a radial pattern. The imaging device 611 may capture a 360 degree radial ultrasound about an axis D. The imaging system 604 may generate image data for a field of view 619. In this example, the imaging localization system 606 may include a shape sensor (e.g., an optical fiber shape sensor) that provides position and orientation information for the distal end portion 613. In this example, a marker 620 may be visible in the field of view 619 to provide a rotational reference point. In some examples, the marker 620 may be a protrusion or an obstruction that may be visible in each two-dimensional constituent image. In some examples, the marker may be omitted, and the orientation of the two-dimensional ultrasound images may be determined based on a structure, such as the target tissue, in the constituent images. For example, the target tissue may have a high contrast area that is identifiable in the series of images. The shape of the target tissue may be generally known from pre-operative imaging and thus may serve as a template for aligning the constituent images.

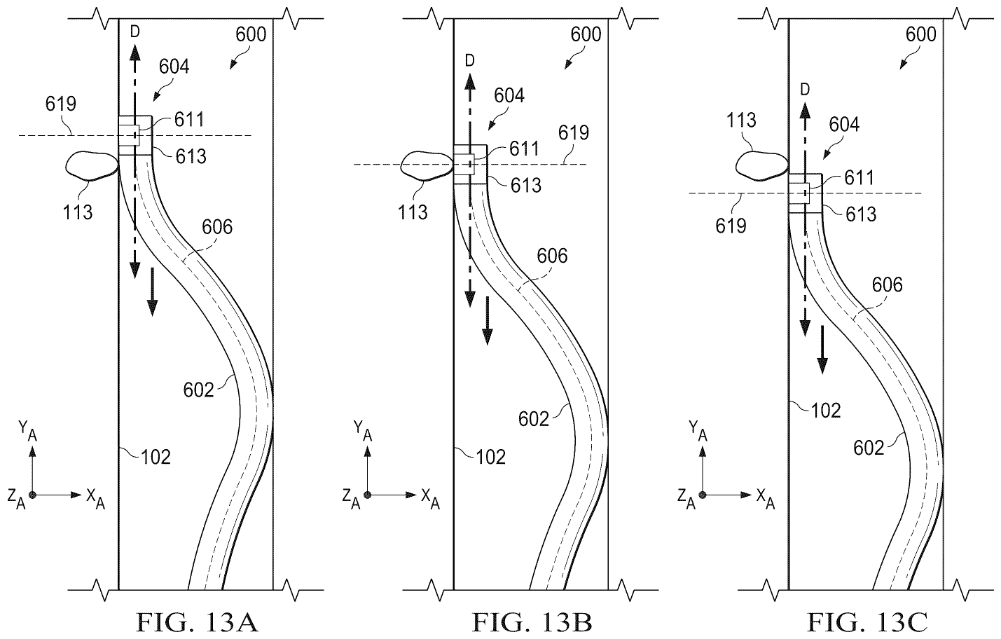

As shown in FIGS. 13A-13C, constituent two-dimensional images used to create a composite three-dimensional image may be generated from the sensing instrument 602 in a series of imaging configurations. As shown in FIG. 13A, the distal end portion 613 and the imaging device 611 of the sensing instrument 602 may be located at a first longitudinal position within the passageway 102 and at a first location along a longitudinal axis D (e.g. in a Y_{A} direction). While at the first longitudinal position, a plurality of image data sets may be obtained by capturing image data from the imaging device 611 and localization data from the shape sensor of the imaging localization system 406 at different imaging configurations, while the imaging device 611 is rotated through a series of orbital (e.g., 360 degree) rotational positions about the longitudinal axis D. Localization data may be obtained from the shape sensor of the imaging localization system 506 (e.g. that provides position and orientation data for the distal end portion) and from the imaging device (e.g., that provides imaging device rotation orientation information). As shown in FIG. 13B, using an instrument pull-back technique, the distal end portion 613 of the sensing instrument 502 may be translated in a generally −Y_{A} direction to a second longitudinal position within the passageway 102. While at the second longitudinal position, a plurality of image data sets may be obtained by capturing image data and localization data while the imaging device 611 is rotated through a series of orbital positions about the longitudinal axis D. As shown in FIG. 13C, the distal end portion 613 of the sensing instrument 602 may be translated further in a generally −Y_{A} direction to a third longitudinal position within the passageway 102. While at the third longitudinal position, a plurality of image data sets may be obtained by capturing image data and localization data while the imaging device 611, is rotated through a series of orbital positions about the longitudinal axis D. The translation of the distal end portion 613 and the imaging device 611 may continue until the full target tissue site and any margin areas are imaged. The image data sets obtained at the various longitudinal positions and rotational orientations may be used to generate a three-dimensional image of the target tissue 113 and surrounding anatomic area. More specifically, the two-dimensional images obtained at the various longitudinal positions and rotational orientations may be stitched together based at least on the localization data captured for each of the configurations to generate a three-dimensional image of the target tissue 113 and the surrounding anatomic area.

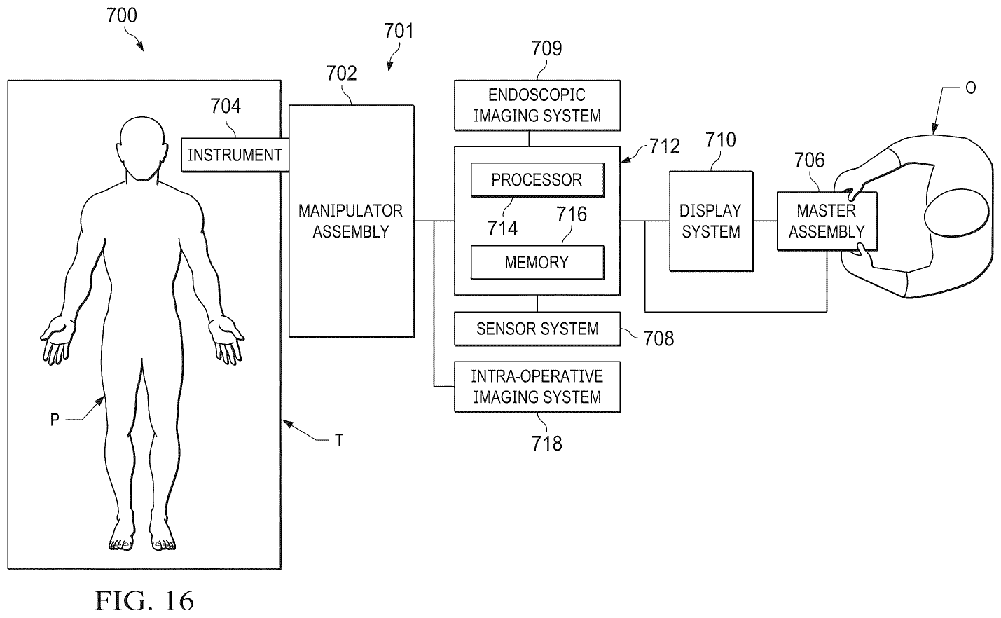

In some examples, medical procedures may be performed using hand-held or otherwise manually controlled medical instrument systems of this disclosure. In other examples, the described medical instrument systems or components thereof may be manipulated with a robot-assisted medical system as shown in FIG. 16. FIG. 16 illustrates a robot-assisted medical system 700. The robot-assisted medical system 700 generally includes a manipulator assembly 1102 for operating a medical instrument system 704 (including, for example, medical instrument system 100, 150, 400, 500, 600) in performing various procedures on a patient P positioned on a table T in a surgical environment 701. The manipulator assembly 702 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 706, which may be inside or outside of the surgical environment 701, generally includes one or more control devices for controlling manipulator assembly 702. Manipulator assembly 702 supports medical instrument system 1104 and may include a plurality of actuators or motors that drive inputs on medical instrument system 704 in response to commands from a control system 712. The actuators may include drive systems that when coupled to medical instrument system 704 may advance medical instrument system 704 into a naturally or surgically created anatomic orifice. Other drive systems may move the distal end of medical instrument system 704 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulatable end effector of medical instrument system 704 for grasping tissue in the jaws of a biopsy device and/or the like.

Robot-assisted medical system 700 also includes a display system 710 (which may display, for example, constituent ultrasound image generated by the sensing instrument or the composite three-dimensional image) for displaying an image or representation of the interventional site and medical instrument system 704 generated by a sensor system 708, an intra-operative imaging system 718, and/or an endoscopic imaging system 709. Display system 710 and master assembly 706 may be oriented so operator O can control medical instrument system 704 and master assembly 706 with the perception of telepresence.

In some examples, medical instrument system 704 may include components for use in surgery, biopsy, ablation, illumination, irrigation, or suction. In some examples, medical instrument system 704 may include components of the endoscopic imaging system 709, which may include an imaging scope assembly or imaging instrument (e.g. a visible light and/or near infrared light imaging) that records a concurrent or real-time image of a interventional site and provides the image to the operator or operator O through the display system 710. The concurrent image may be, for example, a two or three-dimensional image captured by an imaging instrument positioned within the interventional site. In some examples, the endoscopic imaging system components may be integrally or removably coupled to medical instrument system 704. However, in some examples, a separate endoscope, attached to a separate manipulator assembly may be used with medical instrument system 704 to image the interventional site. The endoscopic imaging system 709 may be implemented as hardware, firmware, software, or a combination thereof which interact with or are otherwise executed by one or more computer processors, which may include the processors of the control system 712.

The sensor system 708 may include a position/location sensor system (e.g., an electromagnetic (EM) sensor system) and/or a shape sensor system for determining the position, orientation, speed, velocity, pose, and/or shape of the medical instrument system 704. The imaging localization systems described herein may include all or portions of the sensor system 708.

Robot-assisted medical system 700 may also include control system 712. Control system 712 includes at least one memory 716 and at least one computer processor 714 for effecting control between medical instrument system 704, master assembly 706, sensor system 708, endoscopic imaging system 709, intra-operative imaging system 718, and display system 710. Control system 712 also includes programmed instructions (e.g., a non-transitory machine-readable medium storing the instructions) to implement some or all of the methods described in accordance with aspects disclosed herein, including instructions for providing information to display system 710.

Control system 712 may further include a virtual visualization system to provide navigation assistance to operator O when controlling medical instrument system 704 during an image-guided interventional procedure. Virtual navigation using the virtual visualization system may be based upon reference to an acquired pre-operative or intra-operative dataset of anatomic passageways. The virtual visualization system processes images of the interventional site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intra-operative imaging system 718 may be arranged in the surgical environment 701 near the patient P to obtain images of the anatomy of the patient P during a medical procedure. The intra-operative imaging system 718 may provide real-time or near real-time images of the patient P. In some examples, the intra-operative imaging system 718 may comprise an ultrasound imaging system (e.g. imaging system 154, 404, 504, 604) for generating two-dimensional and/or three-dimensional ultrasound images. For example, the intra-operative imaging system 718 may be at least partially incorporated into sensing instrument 152. In this regard, the intra-operative imaging system 718 may be partially or fully incorporated into the medical instrument system 704.

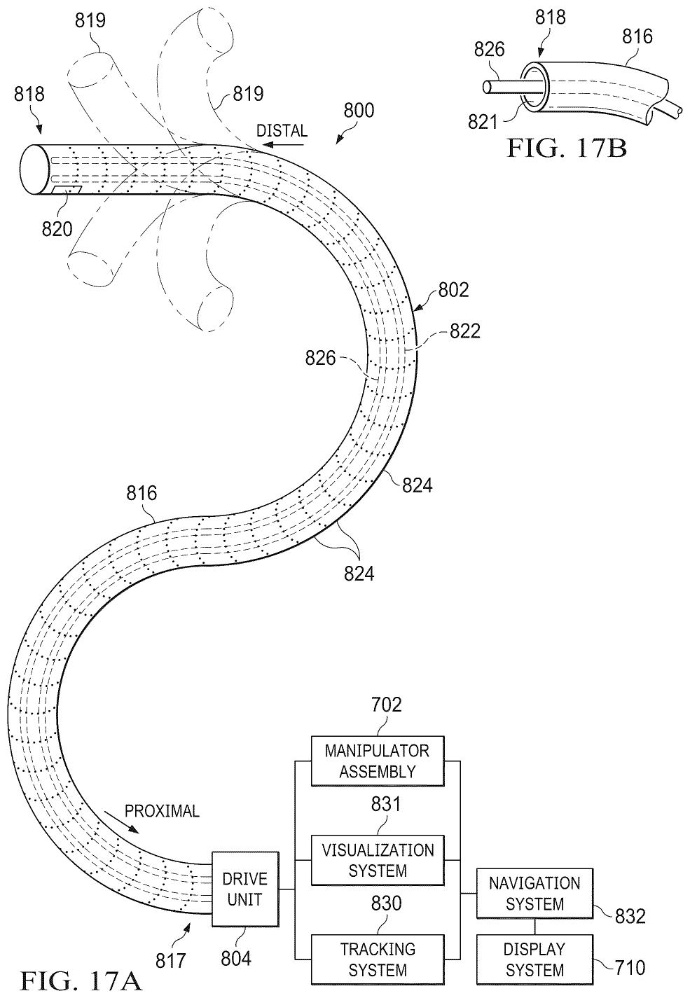

FIG. 17A is a simplified diagram of a medical instrument system 800 according to some examples. The medical instrument system 800 may be used as medical instrument 704 in an image-guided medical procedure performed with robot-assisted medical system 700. In some examples, the medical instrument system 800 may be or include any of the sensing instruments described above (e.g. sensing instrument 152, 402, 502, 602). In some examples, medical instrument system 800 may be used for non-robotic exploratory procedures or in procedures involving traditional manually operated medical instruments, such as endoscopy.

Medical instrument system 800 includes elongate device 802, such as a flexible catheter, coupled to a drive unit 804. Elongate device 802 includes a flexible body 816 having proximal end 817 and distal end, or tip portion, 818. In some embodiments, flexible body 816 has an approximately 3 mm outer diameter. Other flexible body outer diameters may be larger or smaller.

Medical instrument system 800 further includes a tracking system 830 for determining the position, orientation, speed, velocity, pose, and/or shape of distal end 818 and/or of one or more segments 824 along flexible body 816 using one or more sensors and/or imaging devices as described in further detail below. The entire length of flexible body 816, between distal end 818 and proximal end 817, may be effectively divided into segments 824. Tracking system 830 may optionally be implemented as hardware, firmware, software or a combination thereof which interact with or are otherwise executed by one or more computer processors, which may include the processors of control system 712 in FIG. 16.

Tracking system 830 may optionally track distal end 818 and/or one or more of the segments 824 using a shape sensor 822. Shape sensor 822 may optionally include an optical fiber aligned with flexible body 816 (e.g., provided within an interior channel (not shown) or mounted externally). In one embodiment, the optical fiber has a diameter of approximately 200 μm. In other embodiments, the dimensions may be larger or smaller. The tracking system 830 may include the any of the imaging localization systems described above (e.g., imaging localization system 156). The optical fiber of shape sensor 822 forms a fiber optic bend sensor for determining the shape of flexible body 816. In one alternative, optical fibers including Fiber Bragg Gratings (FBGs) are used to provide strain measurements in structures in one or more dimensions. Sensors in some embodiments may employ other suitable strain sensing techniques, such as Rayleigh scattering, Raman scattering, Brillouin scattering, and Fluorescence scattering. In some embodiments, the shape of the elongate device may be determined using other techniques. In some embodiments, tracking system 830 may optionally and/or additionally track distal end 818 using a position sensor system 820, such as an electromagnetic (EM) sensor system. An EM sensor system may include one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 820 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 816 includes a channel 821 sized and shaped to receive a medical instrument 826 including any of the interventional tools described above (e.g., interventional tool 166). FIG. 17B is a simplified diagram of flexible body 816 with medical instrument 826 extended according to some embodiments. In some embodiments, medical instrument 826 may be used for procedures such as surgery, biopsy, ablation, illumination, irrigation, or suction. Medical instrument 826 can be deployed through channel 821 of flexible body 816 and used at a target location within the anatomy. Medical instrument 826 may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. Medical instrument 826 may be advanced from the opening of channel 821 to perform the procedure and then retracted back into the channel when the procedure is complete. Medical instrument 826 may be removed from proximal end 817 of flexible body 816 or from another optional instrument port (not shown) along flexible body 816.

Medical instrument 826 may additionally house cables, linkages, or other actuation controls (not shown) that extend between its proximal and distal ends to controllably the bend distal end of medical instrument 826. Flexible body 816 may also house cables, linkages, or other steering controls (not shown) that extend between drive unit 804 and distal end 818 to controllably bend distal end 818 as shown, for example, by broken dashed line depictions 819 of distal end 818. In some examples, at least four cables are used to provide independent “up-down” steering to control a pitch of distal end 818 and “left-right” steering to control a yaw of distal end 881. In embodiments in which medical instrument system 800 is actuated by a robot-assisted assembly, drive unit 804 may include drive inputs that removably couple to and receive power from drive elements, such as actuators, of the robot-assisted assembly. In some embodiments, medical instrument system 800 may include gripping features, manual actuators, or other components for manually controlling the motion of medical instrument system 800.

In some embodiments, medical instrument system 800 may include a flexible bronchial instrument, such as a bronchoscope or bronchial catheter, for use in examination, diagnosis, biopsy, or treatment of a lung. Medical instrument system 800 is also suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like. The information from tracking system 830 may be sent to a navigation system 832 where it is combined with information from visualization system 831 and/or the preoperatively obtained models to provide the physician or other operator with real-time position information. In some examples, the real-time position information may be displayed on display system 710 of FIG. 16 for use in the control of medical instrument system 800. In some examples, control system 716 of FIG. 1 may utilize the position information as feedback for positioning medical instrument system 800.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 712) or may be implemented, at least in part, in the form of executable code stored on non-transitory, tangible, machine-readable media that when run by one or more processors (e.g., the processors 714 of control system 712) may cause the one or more processors to perform one or more of the processes.

One or more elements in examples of this disclosure may be implemented in software to execute on a processor of a computer system such as control processing system. When implemented in software, the elements of the examples may be the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples described herein are not described with reference to any particular programming language. It will be appreciated that a variety of programming languages may be used to implement the teachings as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

While certain illustrative examples have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

1. A system comprising:
an elongate flexible instrument including an imaging device disposed at a distal portion of the elongate flexible instrument and a localization sensor within the elongate flexible instrument; and
a controller comprising one or more processors configured to:
capture a first two-dimensional image with the imaging device in a first imaging configuration,
receive first localization data for the distal portion of the elongate flexible instrument from the localization sensor while the imaging device is in the first imaging configuration,
create a first image data set including the first localization data and the first two-dimensional image,
capture a second two-dimensional image with the imaging device in a second imaging configuration,
receive second localization data for the distal portion of the elongate flexible instrument from the localization sensor while the imaging device is in the second imaging configuration,
create a second image data set including the second localization data and the second two-dimensional image, and
generate a three-dimensional image based on a plurality of image data sets, including the first and second image data sets.

2. The system of claim 1, wherein the imaging device comprises an ultrasound imaging device.

3. The system of claim 2, wherein the ultrasound imaging device includes an imaging array of ultrasound transducers.

4. The system of claim 3, wherein the imaging array extends along a side wall of the elongate flexible instrument.

5. The system of claim 4, wherein the imaging array comprises a linear phased array.

6. The system of claim 4, wherein the imaging array comprises a curved phased array.

7. The system of claim 3, wherein the imaging array is arranged on a distal end surface of the elongate flexible instrument.

8. The system of claim 7, wherein the imaging array comprises a linear phased array.

9. The system of claim 7, wherein the imaging array comprises an annular array.

10. The system of claim 2, wherein the ultrasound imaging device includes a radial transducer.

11. The system of claim 1, wherein the elongate flexible instrument further includes a tool channel.

12. The system of claim 11, further comprising an interventional tool extendable through the tool channel.

13. The system of claim 11, wherein the tool channel terminates on a side wall of the elongate flexible instrument.

14. The system of claim 11, wherein the tool channel terminates at a distal end face of the elongate flexible instrument.

15. The system of claim 1, wherein the one or more processors are further configured to move the imaging device from the first imaging configuration to the second imaging configuration.

16-17. (canceled)

18. The system of claim 1, wherein the one or more processors are further configured to register the three-dimensional image to a pre-operative model.

19. The system of claim 1, wherein the one or more processors are further configured to display the three-dimensional image with a real-time image of an interventional procedure.

20. The system of claim 1, wherein the elongate flexible instrument further includes an optical imaging system.

21-22. (canceled)

23. The system of claim 1, wherein the one or more processors are further configured to display a two-dimensional guidance image to guide positioning of the elongate flexible instrument.

24. (canceled)

25. The system of claim 1, wherein the one or more processors are further configured to:
capture a third two-dimensional image with the imaging device;
receive a user selection of a reference marker on the third two-dimensional image; and transform the third two-dimensional image and the reference marker into a three-dimensional coordinate space.

26-61. (canceled)
